(19) United States
(12) Patent Application Publication
(10) Pub. No.: US 2018/0236228 A1
(43) Pub. Date: Aug. 23, 2018

(54) PERSONAL RECOVERY AND PERFORMANCE SYSTEM

(71) Applicant: PAUL MATTHEWS, JR., Vernon Hills, IL (US)

(72) Inventor: PAUL MATTHEWS, JR., Vernon Hills, IL (US)

(21) Appl. No.: 15/439,138

(22) Filed: Feb. 22, 2017

(51) Int. Cl. A61N 1/36; A61B 5/00

(52) U.S. Cl. CPC A61N 1/36003; A61B 5/4519

(57) ABSTRACT

An electrical muscle stimulation (EMS) device and a method for using it are disclosed herein. The EMS device includes at least one electrode, a microcontroller, a buffer, a DAC, and an amplifier. The method includes placing at least one electrode in contact with the skin of a patient, performing a diagnostic to determine the soft tissue injury, securing the at least one electrode to the patient, turning on the EMS device, and performing movement protocols. The EMS device transmits pulses to the soft tissue injury using a waveform. The waveform has an alternating polarity.

### FIELD OF INVENTION

This application is in the field of electro-therapeutic stimulation.

### BACKGROUND

Currently electrical muscle stimulation is an accepted and commonly used modality by clinicians around the world including, but not limited to, physiotherapists, physical therapists, chiropractors, athletic trainers, acupuncturists, and osteopaths, as well as by patients themselves. The electrical muscle stimulation is used for treating pain, stimulating muscles, and helping the body to heal faster and more efficiently from soft tissue injuries.

Muscles by nature absorb force. If a patient has pain in one area of the body, the overwhelming majority of the time where pain is felt and essentially where the origin of that pain is coming from are not the same place. The theory is embedded in the idea that force traveling through the body will trigger a nervous system reaction to fire the muscles and absorb the force. When the body has, for whatever reason, resorted to a compensational strategy to handle the force by using the muscles inefficiently often results in poor biomechanics and ultimately an injury of some kind. The pain is felt where something finally got torn, pulled, strained, fractured, and the like. However, the origin of that injury may be embedded in a muscular breakdown. In order to ultimately fix the long term, pain free, optimal range of motion, one must restore sound biomechanics, proper function, and balance to the muscular system. For example, a patient may have pain in the front of the shoulder caused by poor posture and poor biomechanics, specifically weak muscles in the posterior shoulder. The excessive overuse of the anterior structures of the shoulder eventually begins causing discomfort/unnatural wear and tear and pain in the front of the shoulder.

There are a large number of electrical muscle stimulation devices currently in the market place. There are several disadvantages to similar devices including cost, accessibility, and portability. There is a need for an electrical muscle stimulation device that is cheaper, accessible, and portable.

### SUMMARY

An electrical muscle stimulation (EMS) device providing a revolutionary approach to rehabilitating soft tissue injuries is disclosed herein. The EMS device may have unique features including a diagnostic search function and cutting edge wave form technology.

The diagnostic search function of the EMS device includes one rubber electrode/sponge placed as a ground connection, away from the area in search, and a second rubber electrode/sponge used to search over other areas of the body to determine where there is a neuromuscular imbalance. The created waveform may be a combination of different variables, including pulses/second, shape, current, and polarity.

An electrical muscle stimulation (EMS) device for producing a waveform that is utilized in rehabilitating soft tissue injuries is disclosed herein. The EMS device includes at least one electrode, a microcontroller, a buffer, wherein the buffer comprises the waveform, a digital to analog converter (DAC), and an amplifier. The waveform has an alternating polarity.

A method for using an electrical muscle stimulation (EMS) device to rehabilitate soft tissue injuries is disclosed herein. The method includes placing at least one electrode in contact with the skin of a patient, performing a diagnostic to determine the soft tissue injury, securing the at least one electrode to the patient, turning on the EMS device, wherein the EMS device transmits pulses to the soft tissue injury, and performing movement protocols. The EMS device transmits the pulses using a waveform and the waveform has an alternating polarity.

### BRIEF DESCRIPTION OF THE DRAWINGS

FIG. 1 is an example of an electrical muscle stimulation (EMS) device; and

FIG. 2 is an example of a method for using the EMS device.

### DETAILED DESCRIPTION

This invention is described in the following description with reference to the Figures, in which like reference numbers represent the same or similar elements. While this invention is described in terms of modes for achieving this invention's objectives, it will be appreciated by those skilled in the art that variations may be accomplished in view of these teachings without deviating from the spirit or scope of the present invention. The embodiments and variations of the invention described herein, and/or shown in the drawings, are presented by way of example only and are not limiting as to the scope of the invention.

Unless otherwise specifically stated, individual aspects and components of the invention may be omitted or modified, or may have substituted therefore known equivalents, or as yet unknown substitutes such as may be developed in the future or such as may be found to be acceptable substitutes in the future. The invention may also be modified for a variety of applications while remaining within the spirit and scope of the claimed invention, since the range of potential applications is great, and since it is intended that the present invention be adaptable to many such variations.

An electrical muscle stimulation (EMS) device providing a revolutionary approach to rehabilitating soft tissue injuries is disclosed herein. The EMS device may identify and repair the motor innervation of weakened soft tissue structures by helping locate the effected tissues and restore optimal motor innervation to compromised neuromuscular tissues; most notably when combined with the proper therapy and device assisted movement protocols. The EMS device may have unique features including a diagnostic search function and cutting edge wave form technology.

The diagnostic search function of the EMS device includes one rubber electrode/sponge placed as a ground connection, away from the area in search, and a second rubber electrode/sponge used to search over other areas of the body to determine where there is a neuromuscular imbalance.

Muscles by nature absorb force and the waveform technology described herein has the ability to determine what areas of the nervous system have fallen out of neuromuscular balance. The waveform technology gives the user an uncomfortable/intensified sensation when the EMS device is set to a specified power output and pulses per second rate. Using the designed waveform, rubber electrodes with sponges are moved in close contact with the skin on most areas of the body to determine pad placement.

If a patient has pain in one area of the body, an overwhelming majority of the time where the pain is felt and where the origin of that pain is coming from are not the same place. This theory is embedded in the idea that force traveling through the body will trigger a nervous system reaction to fire the muscles and absorb the force. When the body, for whatever reason, resorts to a compensational strategy to handle the force, it uses the muscles inefficiently. This often results in poor biomechanics and ultimately an injury of some kind. The pain is felt where something finally got torn, pulled, strained, fractured, and the like. However, the origin of that injury may be embedded in a muscular breakdown. In order to fix the long term pain and restore an optimal range of motion, sound biomechanics, proper function, and balance, the muscular system must be restored to the operating system of the patient's nervous system.

For example, a patient may have pain in the front of the shoulder caused by poor posture and poor bio mechanics; specifically weak muscles in the posterior shoulder. The excessive overuse of the anterior structures of the shoulder eventually starts causing discomfort/unnatural wear and tear and pain in the front of the shoulder. The search electrode and sponge, disclosed herein, may probe over the posterior shoulder. The patient may experience a noticeable discomfort when the electrode is properly placed over the site of the neuromuscular imbalance. This is based on the idea that if a patient is weak in one particular area of the body the brain ultimately defaults to a mechanism in which it will avoid using the weak area and compensates by using a different muscle or synergistic muscle group to sequence the action.

The EMS device not only identifies the area of muscular imbalance, but may help treat and activate the brains ability to reestablish a better neural connection. Once the appropriate area has been determined by the EMS device, the electrodes and sponges are secured in place and may be used with a variety of protocols that can be both passive and active.

The EMS device may be portable. The waveform technology, utilized by the EMS device, allows the user to ultimately perform a number of movements that specifically target an area of weakness. The user, while being hooked up to the EMS device, may perform movements that force the body to use those specific muscles, creating an increase in intensity of the device when the user has bad biomechanics in movement. The EMS device may be used for a wide range of purposes, but specifically for treating a region of the body that's out of neuromuscular balance or is weak in conjunction with endless varying movement protocols.

The EMS device has a highly unique design that combines an unprecedented amount of functionality and power into a very small and portable unit. The EMS device may be safely secured to the body of the user while the user performs any number of movements, all while the EMS device is running and stimulating/activating a targeted area of a muscle or muscle group. The EMS device has a diagnostic function in which it determines the areas out of neuromuscular imbalance (muscle weakness) that need to be treated. This is achieved based on proprietary waveform technology that puts a stimulus into a weak muscle/weak neural connection that is not accustomed to properly absorbing force resulting in a very noticeable increase in discomfort when the device is set accordingly.

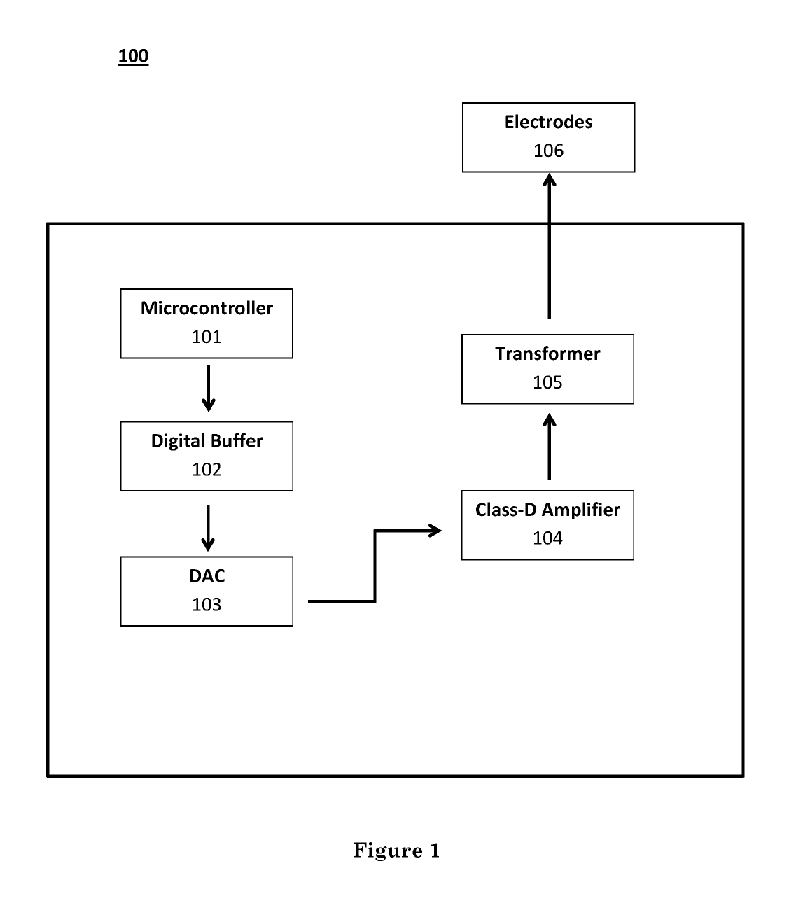

FIG. 1 is an example an electrical muscle stimulation device. The EMS device 100 includes a microcontroller 101, a digital buffer 102, a digital to analog converter (DAC) 103, a class-D amplifier 104, a transformer 105, and probes 106. The microcontroller 101 runs customer firmware that computes a buffer. The buffer contains a digital representation of a desired waveform. The digital data buffer, containing the waveform, is transmitted to the DAC 103. The DAC 103 converts the digital values of the waveform into analog voltages. The analog voltage waveform is fed to the class-D amplifier 104. The class-D amplifier 104 amplifies the signal of the waveform. The amplified waveform signal is then fed to the transformer 105. The transformer 105 boosts the signal voltage. The output of the transformer 105 is transmitted to the electrodes 106 of the EMS device 100.

The created waveform may be a combination of different variables, including pulses/second, shape, current, and polarity. The waveform produces a pulse rate in the range of 1000-2500 pulses/second. Preferably the waveform produces a pulse rate of 2000 pulses/second. The waveform can take on any shape, for example, a sine wave, a square wave, a triangle wave, and a sawtooth wave. Preferably the waveform has sharp square edges. One of the main distinctions of the waveform used in the EMS device is the use of alternating polarities. By alternating the polarity of the waveform, more power can be used. More power results in a more intense pulse for the user, optimizing the end results.

The unique waveform permits movement by the user. In typical devices, the pulses emitted cause essentially a paralyzing effect. With this unique waveform, although more intense, the user has a range of motion that allows for a variety of movements to occur. The user may utilize different movement protocols based on the injury. These movement protocols enable the user to move their bodies in specific ways, causing the muscles in pain to be manipulated to encourage healing.

For example, for shoulder impingement syndrome, a user may engage in manual therapy to release tight and shortened tissue in the anterior structures of the shoulder, including, but not limited to, pectoral major/minor, subscapularis/latissimus dorsi, upper trapezius/neck extensors, and the like. Next, the EMS device searches the posterior shoulder for an area of neuromuscular weakness to determine an area around the rotator cuff, specifically the muscles of the infraspinatus and teres minor region. One electrode and sponge is secured to the low back area as a ground electrode and the other is secured to the rotator cuff using straps to ensure the electrode and sponge are secure. The client user then participates in various banded mobilization stretches to help improve shoulder mobility prior to attempting to strengthen. The user then performs a series of movements that specifically challenge those muscles. For example, the user may perform a standing single arm cable reverse fly, a standing cable Y, a dumbbell External rotation, and a bottom up kettlebell overhead press.

In another example, the EMS device may reveal weakness in the lateral hip abductors. The user may perform a mini band lateral walk, single leg squats, side lying hip abduction, clams with a band, and the like.

In another example, the EMS device may reveal weakness in the medial knee, specifically the vastus medialis oblique causing valgus of the knee in flexion. The user may perform step downs from an adjustable platform, knee dominant split squats, single leg squats, split squats with the front foot on a balance pad for instability, and the like

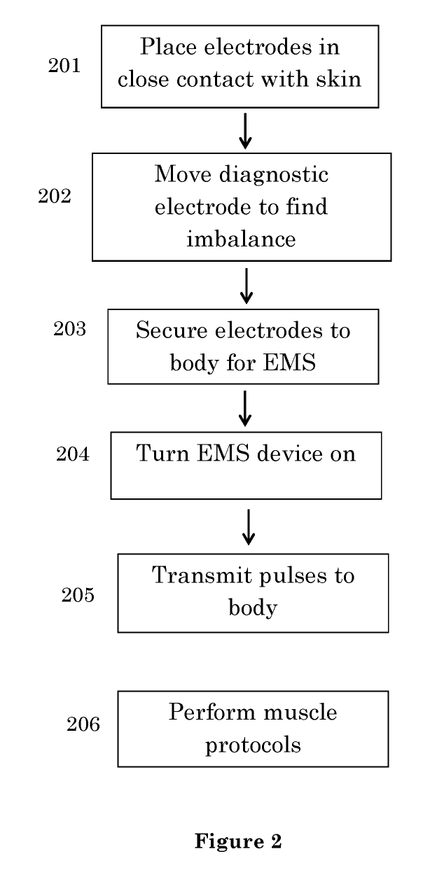

FIG. 2 is an example of a method for using an electrical muscle stimulation device. The electrodes, both a diagnostic electrode and a ground electrode, of the EMS device are put it close contact with the user's skin 201. The diagnostic electrode is moved to find a neuromuscular imbalance 202. Once the neuromuscular imbalance is found, the electrodes are secured to the body 203. The EMS device is then turned on 204 and pulses are sent to the muscles 205. The user performs movement protocols 206 to stimulate the muscles and repair the neuromuscular imbalance.

The EMS device, described herein, provides a more accessible therapy based on its considerably lower price for both rental/lease and ownership terms. The protocols for use align with the most advanced systems for therapy and rehabilitation.

Securing the electrodes to the body is currently an issue when the user engages in more vigorous activity or when the electrodes are placed in certain areas of the body like the back of the shoulder where it's not only difficult to secure them in place but as the user begins to perspire the electrode can move around which can reduce the effect of the therapy and also force the user to have to stop activity and re-secure the electrodes. They have to be securely connected to the user's body otherwise the electrical connection may be lost. Different wraps may be used for different parts of the body to ensure the electrodes are secure. For example, a core/low back strap that is specifically meant to secure electrodes around the knee, similar looking to a knee brace, or a shoulder harness that may secure electrodes in the posterior should and trapezius region.

The EMS device has the ability to revolutionize the rehabilitation, recovery, and performance industries as well as most uses of an electro therapeutic device. The EMS device may be sold to both clinicians as well as end users directly. The device may be used in conjunction with existing treatment platforms or on its own following protocols based on what the diagnostic search reveals. Therefore, for each region of the body, specific protocols exist that the clinician or end user may follow or combine with an endless array of other therapies.

Throughout this disclosure and elsewhere, block diagrams and flowchart illustrations depict methods and apparatuses (i.e., systems). Each element of the block diagrams and flowchart illustrations, as well as each respective combination of elements in the block diagrams and flowchart illustrations, illustrates a function of the methods and apparatuses. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “circuit,” “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a finite sequence of computational instructions or program instructions. It will be appreciated that a programmable apparatus (i.e., computing device) can receive such a computer program and, by processing the computational instructions thereof, produce a further technical effect.

A programmable apparatus includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

In view of the foregoing, it will now be appreciated that elements of the block diagrams and flowchart illustrations support combinations of means for performing the specified functions, combinations of steps for performing the specified functions, program instruction means for performing the specified functions, and so on.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. An electrical muscle stimulation (EMS) device for producing a waveform that is utilized for fixing a neuromuscular imbalance, the EMS device comprising:
at least one electrode;
a microcontroller;
a buffer, wherein the buffer comprises the waveform;
a digital to analog converter (DAC); and
an amplifier;
wherein the waveform has an alternating polarity; and
wherein the waveform is configured to allow movement by a patient of a soft tissue injury.

2. The EMS device of claim 1, wherein the waveform produces a pulse rate in a range of 1000-2500 pulses per second.

3. The EMS device of claim 2, wherein the pulse rate is 2000 pulses per second.

4. The EMS device of claim 1, wherein the waveform has sharp square edges.

5. The EMS device of claim 1, wherein the EMS device is portable.

6. The EMS device of claim 1, wherein the at least one electrode is configured to be secured to the patient.

7. The EMS device of claim 6, wherein the at least one electrode is configured to perform a diagnostic search on the patient to locate the soft tissue injury.

8. The EMS device of claim 7, wherein the at least one electrode is configured to transmit a pulse rate to the soft tissue injury.

9. The EMS device of claim 8, wherein the alternating polarity of the waveform increases the power of the pulses.

10. A method for using an electrical muscle stimulation (EMS) device to rehabilitate soft tissue injuries, the method comprising:
placing at least one electrode in contact with the skin of a patient;
performing a diagnostic to determine a soft tissue injury of the patient;
securing the at least one electrode to the patient;
turning on the EMS device, wherein the EMS device transmits pulses to the soft tissue injury; and
performing patient movement protocols of the soft tissue injury;
wherein the EMS device transmits the pulses using a waveform; and
wherein the waveform has an alternating polarity.

11. The method of claim 10, wherein the pulses have a pulse rate in a range of 1000-2500 pulses per second.

12. The method of claim 11, wherein the pulse rate is 2000 pulses per second.

13. The method of claim 10, wherein the waveform has sharp square edges.

14. The method of claim 10, wherein the EMS device is portable.

15. The EMS device of claim 8, wherein the waveform is configured to allow a range of motion for the patient of the soft tissue injury while the EMS device is producing the waveform on the soft tissue injury.

16. The method of claim 10, wherein the patient movement protocols include movement by the patient of the soft tissue injury.

17. The method of claim 16, wherein the movement by the patient of the soft tissue injury occurs while the EMS device transmits pulses to the soft tissue injury.

18. The method of claim 10, wherein the alternating polarity of the waveform increases the power of the pulses.

19. A method for using an electrical muscle stimulation (EMS) device to rehabilitate soft tissue injuries, the method comprising:
placing at least one electrode in contact with the skin of a patient;
performing a diagnostic to determine the soft tissue injury;
securing the at least one electrode to the patient; and
turning on the EMS device, wherein the EMS device transmits pulses to the soft tissue injury;
wherein the EMS device transmits the pulses using a waveform;
wherein the waveform has an alternating polarity; and
wherein the patient moves the soft tissue injury while the EMS device transmits pulses to the soft tissue injury.

20. The method of claim 19, wherein the soft tissue injury is in one of a posterior shoulder area of the patient, a lateral hip abductor area of the patient, or a medial knee area of the patient.
